(19) United States
(12) Patent Application Publication
(10) Pub. No.: US 2002/0004206 A1
(43) Pub. Date: Jan. 10, 2002

(54) Methods of screening for disease

(76) Inventors: Barry M. Berger, Chestnut Hill, MA (US); Steven J. Laken, Pepperell, MA (US); Stanley N. Lapidus, Bedford, NH (US)

Correspondence Address: TESTA, HURWITZ & THIBEAULT, LLP, HIGH STREET TOWER, 125 HIGH STREET, BOSTON, MA, 02110, US

(21) Appl. No.: 09/859,990

(22) Filed: May 17, 2001

Related U.S. Application Data
(63) Continuation-in-part of application No. 09/545,162, filed on Apr. 7, 2000.
(60) Provisional application No. 60/128,629, filed on Apr. 9, 1999.

(30) Foreign Application Priority Data
00000000

(51) Int. Cl. C12Q 1/68

(52) U.S. Cl. 435/6

(57) ABSTRACT

The invention provides methods of screening for disease in a patient by performing a non-invasive or minimally invasive genetic assay on a sample from the patient to detect characteristics indicative of the presence of a disease in the sample at a predetermined time following the performance of a diagnostic procedure on a patient to detect characteristics indicative of the presence of a disease. Methods of this invention are useful in screening for cancer.

### RELATED APPLICATIONS

This application is a continuation in part of U.S. application Ser. No. 09/545,162, which claims the benefit of No. 60/128,629, the disclosure of each of which is incorporated by reference herein.

### FIELD OF THE INVENTION

This invention generally relates to methods of screening for disease in a patient, and more specifically to methods of screening for disease by performing a non-invasive genetic assay on a sample at a predetermined time following the performance of a diagnostic procedure on a patient. Methods of the invention are especially useful in screening for cancer.

### BACKGROUND OF THE INVENTION

Current methods of disease screening involve examining or testing individuals for early stages of disease. Preferably, individuals are screened for disease even before they exhibit symptoms. Early-stage screening is important because early diagnosis of a disease can make treatment easier and more effective, and can decrease mortality. Additionally, early treatment of a disease may help to slow, stop, or even reverse disease progression so that an individual never becomes symptomatic.

Many diseases are curable if detected early in their development. Colorectal cancer is an example of a disease that is highly curable when diagnosed early. Colorectal cancer typically originates in the colonic epithelium and is not extensively vascularized (and therefore not invasive) during the early stages of development. The transition to a highly-vascularized, invasive and ultimately metastatic cancer commonly takes ten years or longer. With early detection and diagnosis, colon cancer may be effectively treated by, for example, surgical removal of the cancerous or precancerous tissue. However, colorectal cancer is often detected only upon manifestation of clinical symptoms, such as pain and black tarry stool. Generally, such symptoms are present only when the disease is well established, and only after metastasis has occurred. Early detection of colorectal cancer is therefore important in order to significantly reduce its morbidity.

Generally, the risk of developing cancer increases with age. For example, the death rate from a colon carcinoma increases more than 1000-fold between the ages of thirty and eighty. Cooper, Oncogenes, 4-6 (2d ed. 1995). As a result, all individuals age fifty and above in the United States are recommended to undergo some form of colorectal cancer screening on a regular basis. Those individuals with a greater risk for the development of colorectal cancer should be screened at earlier ages and more frequently. Other risk factors for colorectal cancer include familial and genetic factors. Currently, the best means of preventing colorectal cancer is through early detection of pre-neoplastic lesions in the colon through various invasive and noninvasive screening techniques.

The current recommended screening techniques for colorectal cancer, which include flexible sigmoidscopy and colonscopy repeated at various intervals, have a number of limitations: 1) the time interval between development of polyps or other pre-neoplastic lesions is long, which requires continued periodic screening for colorectal cancer; 2) the repetitive application of invasive procedures to individuals who are only at increased risk for colon cancer because of advanced age alone subjects patients to the inherent risks and discomfort associated with such procedures; and 3) despite the availability of the current recommended screening techniques for colorectal cancer, the screening techniques are vastly underused, due in large part to patient non-compliance.

Most methods for screening for cancer are invasive. Invasive diagnostic screening methods, such as endoscopic examination, allow for direct visual identification, removal, and biopsy of potentially-cancerous tissue. However, invasive cancer screening procedures are often expensive, inherently risky, and can result in severe medical complications. Invasive screening procedures also frequently result in significant patient discomfort. The discomfort associated with typical invasive screening methods reduces patient compliance with routine screening procedures. For example, flexible sigmoidoscopy is an invasive procedure for diagnosing colorectal cancer that enables detection of approximately 55% of all colorectal cancer and is estimated to have an 85% sensitivity with a near 100% specificity. However, the procedure has a complication rate of 4.5 per 10,000 persons screened. Eddy, Ann. Intern. Med., 113: 373-384 (1990). Further, patient compliance with physicians' recommendations to undergo sigmoidoscopy is low, reportedly varying from 30 to 75%, due to discomfort and perceived embarrassment associated with this procedure. Kelly et al., Med. Care, 30: 1029-1042 (1992); McCarthy et al., J. Gen. Intern. Med., 8: 120-125 (1993).

Non-invasive methods of screening for cancer involve assaying samples for the presence of materials that are indicative of cancer or pre-cancer. Established non-invasive methods for detection of colorectal cancer focus on extracellular indicia of the presence of cancer, such as the presence of fecal occult blood or elevated levels of carcinoembryonic antigen, both of which are suggestive of the presence of colorectal cancer. However, such extracellular indicia typically occur only after the cancer has become invasive, and therefore more difficult to treat. As a result, many non-invasive screening procedures are of limited value in the early diagnosis of cancer. For example, fecal occult blood testing (FOBT) is an non-invasive screening test for colorectal cancer that is highly variable in accuracy, ranging between 28% and 93%, depending upon the subject's hydration status, with a specificity of 96%. Eddy, Ann. Intern. Med., 113: 373-384 (1990). One study estimates, however, that 50 to 60% of all colorectal cancers will be missed if FOBT is the only method of screening used. Allison et al., Ann. Intern. Med., 112: 328-333 (1990).

Alterations in genomic integrity often are associated with disease or with the propensity for disease. For example, sickle cell anemia, phenylketonuria, hemophilia, cystic fibrosis, and various cancers have been associated with one or more genetic mutations. Cancer has been associated with genetic mutations in a number of oncogenes and tumor suppressor genes. Duffy, Clin. Chem., 41: 1410-1413 (1993). Cancer is thought to arise through a series of mutations in genomic DNA, resulting in genomic instability and uncontrolled cellular growth. In normal cells, damage to genomic DNA typically leads to expression of tumor suppressors, such as the cell-cycle regulator, p53. Damage to cellular DNA results in increased expression of p53 which arrests the cell cycle to allow repair of the damage. If the damaged DNA cannot be repaired, the cell undergoes apoptosis. The process of apoptosis is important not only in the regulation of cellular metabolism, but also in inhibiting oncogenesis. As cells undergo apoptosis, the nucleus becomes small and fragmented. Nuclear DNA is digested into spindle fragments that are generally no larger than about 200 base pairs. As the process continues, usually through multiple pathways, the cell membrane breaks down, and cellular contents are metabolized. As a result, cells that have the potential to enter the multi-step pathway leading to cancer are eliminated, thus preventing the accumulation of additional mutations in daughter cells. Unfortunately, however, not all mutations result in apoptosis. For example, if there is a mutation in the p53 gene itself (or in another cell cycle regulator), damaged cells will proceed through the cell cycle, giving rise to progeny in which additional DNA mutations will go unchecked. It is the accumulation of these mutations that is the hallmark of many cancers.

Recent developments in molecular biology provide methods of great potential for detecting the presence of a range of DNA mutations indicative of disease. Point mutations in the ras genes have been shown to convert those genes into transforming oncogenes. Bos et al., Nature, 315: 726-730 (1985). Mutations and the loss of heterozygosity at the p53 tumor suppressor locus, as discussed above, have been correlated with various types of cancer. Ridanpaa et al., Path. Res. Pract., 191: 399-402 (1995); Hollstein et al., Science, 253: 49-53 (1991) The loss or other mutation of the APC and DCC tumor suppressor genes has also been associated with tumor development. Blum, Europ. J. Cancer, 31A: 1369-1372 (1995). It has been suggested that specific mutations might be a basis for molecular screening assays for the early stages of certain types of cancer. See, e.g., Sidransky et al., Science, 256: 102-105 (1992). Accordingly, non-invasive screening assays that are highly sensitive and highly specific for detecting the presence of a range of DNA mutations indicative of cancer have been developed. For instance, the presence of such mutations can be detected in DNA found in stool samples during various stages of colorectal cancer.

In general, one reason that the recommended techniques in the prior art for disease screening have failed to provide satisfactory results for reducing the morbidity associated with such diseases, especially cancer, is that such methods have not addressed an effective combination of available diagnostic procedures and non-invasive genetic assays for the detection of disease.

Accordingly, there is a need in the art for improved screening techniques that will detect characteristics indicative of the presence of disease for the early diagnosis and enhanced prognosis of disease. Such methods are provided herein.

### SUMMARY OF THE INVENTION

The present invention provides improved early-stage diagnosis and disease monitoring by combining a diagnostic procedure followed by one or more non-invasive or minimally invasive genetic assays. Accordingly, the present invention provides methods of screening for disease by performing a non-invasive or minimally invasive genetic assay on a sample to detect characteristics indicative of the presence of a disease in the sample at a predetermined time interval following the performance of a diagnostic procedure. In a preferred embodiment of the invention, methods comprise performing a diagnostic procedure to detect the presence of cancer and then screening for indicia of cancer in a stool sample by performing a non-invasive or minimally invasive genetic assay to detect the presence of cancerous or precancerous cells or cellular debris in a voided stool sample.

Generally, methods of the invention comprise two or more components separated by predetermined time intervals. A first component is a diagnostic examination, such as a sigmoidoscopy, endoscopy, mammography, prostate-specific antigen, carcinoembryonic antigen 1-18, digital rectal examination, breast self-examination, CT scan, virtual colonoscopy, MRI, PET scan, colonscopy, biopsy, surgery, or other means for diagnosis (hereinafter “diagnostic component”). The diagnostic component is preferably a non-genetic diagnostic procedure. In one embodiment of the invention, the diagnostic component is an invasive diagnostic procedure. Invasive procedures for use in the invention may be any invasive or substantially invasive test that is appropriate for diagnosis, such as surgery, biopsy, colonoscopy, flexible sigmoidoscopy, and endoscopy.

A second component of the invention is a non-invasive or minimally invasive genetic assay (hereinafter “non-invasive component”) for a follow-up to the diagnostic procedures. Generally, the non-invasive component of the invention comprises detecting the presence in a biological sample of species-specific nucleic acids indicative of cancer or precancer. Samples comprising such nucleic acids are identified as having indicia of cancer or precancer.

Each component of the methods of the invention may be repeated. Preferably, the non-invasive component is repeated at regular intervals after performance of the diagnostic component. The invention is based on providing non-invasive or minimally invasive follow-up to a diagnostic procedure at a defined time interval. Accordingly, such follow-up eliminates the need to conduct multiple diagnostic procedures.

Non-cancerous (normal) cells undergo apoptosis at regular intervals, or in response to irreparable cell damage. As a result of apoptosis, DNA from normal cells is cleaved into small fragments having about 200 or fewer base pairs, and typically 180 base pairs or less. In contrast, DNA obtained from cancer or precancer cells is much larger than the typical apoptotic fragments. Thus, the presence of large DNA fragments in a sample (e.g. of sloughed colonic epithelium) indicates that there are or were cells in the sample (or the specimen from which it was obtained) that have avoided apoptosis, and its coincidental degradation of DNA. The presence of large DNA fragments represents a positive screen for cancer or precancer.

Non-invasive testing according to the invention preferably comprises genetic assays for identifying indicia of disease in tissue or body fluid samples by identifying non-apoptotic DNA in those samples. In preferred methods of the invention, patients presenting samples having a high proportion of non-apoptotic nucleic acids as determined by the non-invasive methods of the invention are further evaluated for the presence of a tumor, adenoma, or other cancerous or precancerous lesion. In a highly preferred embodiment, the non-invasive component of the invention is a genetic assay for identifying indicia of disease in samples containing exfoliated epithelial cells. It has now been recognized that DNA obtained from exfoliated normal (non-cancerous) cells is different than DNA obtained from exfoliated cancer or precancer cells. Normal exfoliated cells typically have undergone apoptosis, and thus produce cells or cellular debris (depending upon the stage of apoptosis) comprising DNA that has been substantially degraded. Exfoliated cancer or precancer cells typically have not undergone apoptosis, and such cells or their debris, while producing some very small fragments as a result of degradation in the sample, typically also contain a higher proportion of large DNA fragments (compared to those observed in cells or debris from exfoliated normal cells). The difference in DNA integrity between normal and abnormal cells is a marker for the presence of cancer or precancer in a sample comprising exfoliated cells.

In one embodiment, the non-invasive component of the invention comprises detecting in a biological sample one or more DNA fragment(s) of a length that would not be substantially present in noncancerous cells or cellular debris. There is no upper limit on these fragments, as all that is necessary is that the fragment be larger than an apoptotic fragment. Typically, however, fragments indicative of cancer or precancer cells are between about 200 and about 3500 base pairs, and ideally between about 500 and about 2500 base pairs. In a preferred embodiment, gel electrophoresis, affinity chromatography, or mass spectrometry are used to detect large DNA fragments (fragments comprising greater than about 200 base pairs). The presence of large DNA fragments in the sample is indicative of colorectal cancer.

In a preferred embodiment, the non-invasive component of the invention comprises amplifying nucleic acids in a representative stool sample using human-specific primers, and detecting amplicons having greater than about 200, and preferably about 500 or more base pairs. In a highly-preferred embodiment, amplification is accomplished by polymerase chain reaction (PCR) using forward and reverse primers directed against human-specific nucleic acid fragments, and spaced apart to provide a lower limit on the resulting amplicons. Also in a highly-preferred embodiment, primers for PCR are directed against human oncogene or tumor suppressor sequences. Preferred target nucleic acids for PCR primers include p53, Kras, apc, dcc, and other genes known or suspected to be associated with cancer, and especially colorectal cancer. Methods for conducting PCR are provided in U.S. Pat. No. 4,683,202, incorporated by reference herein. The presence of amplicon greater than about 200 base pairs in length is indicative of template nucleic acid in the sample of that length (or longer). According to the non-invasive component of the invention, such long sequences represent a positive screen and are indicative of cancer or precancer.

Preferred biological samples for use in the non-invasive component of the invention include stool, pus and urine. Tissue (e.g., colon, lungs, bladder) in which cells, especially epithelial cells, are exfoliated are most preferred for the non-invasive screening assays of the invention. In such tissues, continuing cellular renewal requires that cells are regularly sloughed after having undergone apoptosis. Accordingly, non-invasive assays of the invention preferably detect large DNA fragments in samples comprising exfoliate. Samples of the exfoliate (tissue or body fluid containing the exfoliated cells) predominantly comprise apoptotic DNA.

Stool is a good sample for exemplification of the non-invasive component of the invention. The colonic epithelium undergoes a continual process of exfoliation. Normal epithelial cells undergo apoptosis, and are sloughed into the lumen of the colon, and onto forming stool. Cells from polyps and tumors are also sloughed onto forming stool. However, cells from polyps or tumors are, by definition, not apoptotic. In the non-invasive component, methods of the invention take advantage of the different characteristics between apoptotic and non-apoptotic cells in order to screen patient samples for indicia of cancer or precancer.

Preferred non-invasive assays of the invention for use on a stool sample comprise obtaining a representative stool sample. An especially-preferred method for preparing a stool sample is disclosed in U.S. Pat. No. 5,741,650, and in co-owned, co-pending U.S. patent application Ser. No. 09/059,713, each of which is incorporated by reference herein.

Preferred non-invasive assays of the invention further comprise enriching samples used in the non-invasive component for human DNA. Preferred enrichment methods for use in the invention include enriching a desired human target sequence using an affinity column, sequence-specific capture, or through the use of preferred buffers that bias isolation of human DNA. A preferred enrichment method is based upon the capture of unique human nucleic acids using, for example, an affinity column.

In a preferred embodiment, the non-invasive component further comprises the step of extracting DNA from the homogenized sample mixture using sequence-specific nucleic acid probes. Particularly preferred are probes hybridizing to human DNA. The probes are preferably labeled. Preferred labels include radioactive labels, fluorescent labels, molecular weight labels and enzymatic labels. Other labels are well known in the art.

In a preferred embodiment, capture probes comprise DNA, RNA or PNA, and are detectably labeled using methods known in the art. In one embodiment, probes are labeled with radioactive isotopes such as ³²p, ³³p, ³⁵S, ¹²⁵I, or any other detectable isotope useful for labeling a hybridization probe. In an another embodiment, probes are labeled with fluorescent molecules. Numerous fluorescent labels are known in the art, and any detectable fluorescent probe label is useful for practice of the invention. Alternatively, probes are attached to moieties which increase their molecular weight. For example, a probe may be directly attached to a glycoprotein, or a glass bead, or any compound which has a detectable effect on the molecular weight of the probe. In a further embodiment, probes are labeled with a compound that is detectable due to specific interactions with an additional compound. For example, biotinylated probes are detectable via interaction with streptavidin. The streptavidin moiety is attached to a detectable label such as a bead, a fluorescent tag, or an enzyme. In another example, the probes are labeled with a hapten or other antigen which is specifically recognized by an antibody. The antibody is made detectable using methods known in the art including radioactive isotopes, fluorescent tags, and enzyme reactions. In a further example, the probes are directly attached to an enzyme which is detectable via a specific enzyme catalyzed reaction generating a detectable product.

In one embodiment, methods of the invention provide for detection of cancer or pre-cancer in a patient by performing a non-invasive or minimally invasive genetic assay on a sample from the patient to detect characteristics indicative of the presence of cancer or precancer in a sample at a predetermined time interval following the performance of a diagnostic procedure on a patient to detect characteristics indicative of the presence of colorectal cancer. Methods of the invention also provide for monitoring of the disease in a patient currently undergoing treatment or having undergone treatment for cancer.

In a highly preferred embodiment, methods of the invention comprise performing a colonscopy or flexible sigmoidoscopy and then screening for indicia of colorectal cancer in a stool sample by performing a non-invasive or minimally invasive genetic assay to detect DNA resulting from the presence of cells or cellular debris from a cancerous or precancerous lesion into a voided stool sample. In a most preferred embodiment, the non-invasive or minimally invasive genetic assay of the invention is repeated at predetermined time intervals following performance of the diagnostic procedure.

Further aspects and advantages of the invention are contained in the following detailed description thereof.

### DETAILED DESCRIPTION OF THE INVENTION

Methods of the invention are useful as general disease screening techniques, and are useful as screens for a wide-range of disease states. Methods of the inventions are also useful as screening techniques for the presence of cancer and pre-cancer, and are especially useful as screening techniques colorectal cancer and pre-cancer. In addition to colorectal cancers, methods of the invention are useful to screen for other cancers, for example, as screening techniques for lymphomas, stomach cancers, lung cancers, liver cancers, pancreas cancers, prostrate cancers, kidney cancers, testicular cancers bladder cancers, gallbladder cancers, uterine cancers, and ovarian cancers. Methods of the invention are also useful for screening for the presence of cancerous or precancerous lesions in a patient, including adenomas. Adenomas are non-metastatic lesions that frequently have the potential for metastasis. In addition to cancer, methods of the invention are useful, for example, as screening techniques for diseases such as inflammatory bowel syndrome, respiratory distress syndrome, and others in which the performance of diagnostic procedures followed by the performance of non-invasive or minimally invasive assays at a predetermined time thereafter would be effective in the detection of disease. Furthermore, the methods of the invention are also useful for detecting an indicator of the presence of an infectious agent, including, but not limited to, a virus, bacterium, parasite, or other microorganism.

The diagnostic procedures of the invention are procedures performed on a patient to detect characteristics indicative of the presence of a disease. Such diagnostic procedures include, for example, but are not limited to, sigmoidoscopy, mammography, prostate-specific antigen, carcinoembryonic antigen 1-18, digital rectal examination, breast self-examination, CT scan, virtual colonoscopy, MRI, PET scan, colonscopy, surgery, biopsy, endoscopy, laparoscopy, and endoscopic retrograde cholangiopancreatography (ERCP). Diagnostic procedures of the invention also include invasive or substantially invasive diagnostic procedures, and are preferably non-genetic based. While invasive procedures may be effective in diagnosing disease, these procedures are often expensive, inherently risky, and can result in significant patient discomfort and possible medical complications. When such procedures require repetitive application, patient compliance decreases. However, invasive procedures are preferred by many in the medical community for detecting disease, especially cancers.

The non-invasive or minimally invasive genetic assays of the invention are assays conducted on samples obtained non-invasively or minimally invasively from a patient to detect characteristics indicative of the presence of a disease. Such non-invasive or minimally invasive assays of the invention have a high sensitivity for detection of disease when it is present in a patient sample, and a high specificity against false positive results. Such assays include, for example, but are not limited to, DNA analysis of samples from a patient and detection of specific proteins in samples from a patient. The non-invasive or minimally invasive genetic assays of this invention are especially useful for the detection of cancer or pre-cancer in which the assays have a high sensitivity for detection of cancer or pre-cancer when it is present in a patient sample, and a high specificity against false positive results. Non-invasive or minimally invasively assays of the invention include highly-sensitive, highly-specific assays to detect a small amount of a cancer marker (e.g., a DNA mutation) in a sample from a patient. Such non-invasive or minimally invasively assays include, for example, but are not limited to, assays for the detection of mutations at the p53 tumor suppressor locus, in ras genes, in APC and DCC tumor suppressor genes, and in the BAT-26 segment of the MSH2 mismatch repair gene. For purposes of the present invention, a mutation is a deletion, addition, substitution, rearrangement, or translocation in a nucleic acid.

In one aspect of the invention, non-invasive or minimally invasive genetic assays of the invention are based upon the observation that samples comprising cells from patients with cancer or precancer contain a greater amount of high molecular weight (long sequence) DNA fragments as compared to corresponding samples obtained from individuals that are free of cancer/precancer. Accordingly, the non-invasive methods of the invention, following performance of a diagnostic procedure, provide accurate screening procedures for cancer or precancer.

Methods of the invention are useful to detect nucleic acid indicia of cancer or precancer in any tissue or body fluid sample. For example, sputum samples are used to detect the presence of high molecular weight (long sequence) DNA as a marker for cancer. The majority of cells exfoliated into sputum have undergone apoptosis and subsequent further enzymatic degradation. The predominant DNA from those cells is small, apoptotic DNA. Cancer cells produced by, for example, the lungs, the nasal passages, or the trachea will also be sloughed into sputum. However, the DNA from those cells, while being exposed to enzymatic processes, has not been affected by apoptosis. Accordingly, fragments from cancer or precancer cells found in sputum are larger than fragments expected to be produced by normal cells.

Similarly, cells sloughed by cancerous or precancerous lesions in the bladder or kidney produce non-apoptotic DNA in urine, cancerous or precancerous lesions in the lymph nodes result in non-apoptotic DNA fragments in lymph, and cancerous or precancerous cells in the breast slough non-apoptotic DNA-containing cells that can be harvested via aspiration. Accordingly, methods of the invention are useful in any tissue or body fluid. However, for purposes of exemplification of the methods described herein, stool sample were used to predict the presence of colorectal cancer or precancer. Stool is an excellent specimen for analysis due to the characteristic exfoliation of colonic epithelia as described above.

Methods of the invention are practiced by detecting the presence of DNA fragments having a sequence length that would not be expected to be present in significant amounts in a sample obtained from a healthy individual (i.e., an individual who does not have cancer or precancer). A threshold amount of large fragments is an amount that exceeds a predetermined level expected or determined for non-cancerous/non-precancerous cells. The predetermined level or standard can be determined by detecting the amount of a particular size of DNA fragment (preferably apoptotic fragments characteristic of normal cells) in a population or subpopulation of normal patients. Standards can be determined empirically, and, once determined, can be used as the basis for further screening.

The size of fragments to be used is chosen based upon the convenience of the individual performing the screen. Factors affecting the size of fragments used in screening or diagnostic methods of the invention include the availability and costs of probes and primers, the desired target of amplification, the type of cancer being screened, and the patient sample on which screening takes place. The invention takes advantage of the recognition that large fragments exist in greater abundance in abnormal samples than in normal samples. Accordingly, the precise size of fragments used in methods of the invention does not matter. For any given size of fragments to be analyzed, a cutoff must be determined to distinguish between normal and abnormal samples. Preferably, the cutoff is determined empirically based upon known normal and abnormal sample, and then is used in future screenings.

Samples used in the non-invasive component of the invention are biological samples from a patient. Samples from patients are preferably obtained from specimens likely to contain cells or cellular debris. Samples that may be obtained non-invasively or minimally invasively, and thus useful in the methods of the invention include, for example, stool, blood serum or plasma, sputum, pus, saliva, lymph, cerebrospinal fluid, seminal fluid, breast nipple aspirate, biopsy tissue, bile, and pancreatic juice, or urine. However, the skilled artisan recognizes that methods of the invention can be practiced using a variety of different samples in order to detect a variety of cancers, pre-cancers, and other diseases and disorders.

Methods of the invention are useful for early detection of disease, and are especially useful for early detection of cancer or pre-cancer. Methods of the invention are also useful to monitor a patient's response to treatment. The methods of the invention are useful for monitoring cancer in a patient currently undergoing or having undergone treatment for cancer, and are especially useful for monitoring colorectal cancer in a patient currently undergoing or having undergone treatment for colorectal cancer. For example, a diagnostic procedure is performed on an patient and a positive result for disease obtained. The patient undergoes treatment and periodic non-invasive assays are performed on the patient at regular time intervals to monitor the patient's response to treatment. As is apparent to the skilled artisan, methods of the invention are useful to screen the efficacy of any treatment means.

The non-invasive or minimally invasive genetic assays of this invention can be performed on a sample comprising stool. Stool is an example of a biological sample from a patient that is especially useful in the non-invasive component of the invention. A typical stool sample contains patient nucleic acids, but also contains heterologous nucleic acids, proteins, and other cellular debris consistent with the lytic function of the various nucleases, proteinases, etc. found in the colon. If a patient has a developing tumor or adenoma, for example, cells from the tumor or adenoma will also be sloughed onto stool, and they (or their debris) will contain molecular indicia of disease (e.g., DNA mutations).

The non-invasive component of this invention can be repeated at predetermined time intervals. For example, non-invasive assays can be repeated on a patient following the performance of a diagnostic procedure until a positive test result is obtained. Additionally, noninvasive assays can be repeated on a patient at substantially equal or regular time intervals following the performance of a diagnostic procedure. Such time intervals of the invention include, but are not limited to, monthly, bimonthly, quarterly, bi-annually, annually, and semiannually.

The following examples provide further details of methods according to the invention. For purposes of exemplification, the following examples provide details of the use of methods of the present invention in colorectal cancer detection. Accordingly, while exemplified in the following manner, the invention is not so limited and the skilled artisan will appreciate its wide range of application upon consideration thereof.

### EXAMPLE 1

The BAT-26 segment of the MSH1 mismatch repair locus is useful in the noninvasive phase of the invention, as deletions in BAT-26 have been associated with colorectal cancer or adenomas. Stool specimens are collected from the patient and frozen. Each frozen stool specimen, weighing from 7 to 33 grams, is thawed and homogenized in buffer. The buffer is comprised of 0.5 M Tris, 10 mM NaCl, and EDTA, at a volume to mass ratio of about 7:1, essentially as disclosed in U.S. Application Ser. No. 09/491,093, incorporated by reference herein. The concentration of EDTA is a relevant factor, where a range from about 16 mM EDTA to about 300 mM EDTA is useful. More preferably, from about 100 mM EDTA to about 200 mM EDTA is useful. In this example; 150 mM EDTA is used. Each of the samples is then diluted with additional buffer (not containing EDTA) to a final buffer to stool ratio of 20:1. Each sample is centrifuged, and the supernatant, which carries the active DNA degrading fraction, is removed to a clean tube. The supernatant is collected and treated with sodium dodecyl sulfate and Proteinase K. The DNA in each sample is then prepared by standard techniques. See, e.g., Ausubel et al., Short Protocols in Molecular Biology §§ 2.1-2.4 (3d ed. 1995). Generally, a phenol extraction, a phenol/chloroform extraction, and a phenol extraction is performed prior to isolating the DNA. The isolated DNA is then placed into a standard Tris buffer and the captured DNA is amplified using PCR.

A primer is hybridized to the portion of the BAT-26 locus immediately upstream of the poly-A tract, which consists of 26 adenosines (nucleotides 195-221). Unlabeled deoxythymidine, a mixture of labeled and unlabeled deoxycytosine, and unlabeled dideoxyadenine are added along with polymerase. The primer is extended through the poly-A region. The labeled and unlabelled cytosine is extended for the next three bases (nucleotides 222-224, all guanines in the intact sequence) such that label is incorporated into each extended primer. After the poly-A tract and the three guanines, there exist two thymidines in the intact sequence. Thus, the dideoxyadenosine stops primer extension by addition at the end of a primer that is extended through the poly-A and triguanine regions. Strands are separated, and the length of the strands are observed on a polyacrylamide gel to detect deletions in the poly-A tract. Deletions in the poly-A tract are indicative of colorectal cancer.

### EXAMPLE 2

Detection of DNA fragments of at least 200 base pairs in length are also useful in methods of the invention as the amount of 200 bp or greater DNA in a sample is predictive of cancer or precancer in patients. The samples are screened by hybrid capturing human DNA, and determining the amount of amplifiable DNA having at least 200 base pairs. Samples are prepared as described in Example 1.

Each sample is amplified using forward and reverse primers through 7 loci (Kras, exon 1, APC exon 15 (3 separate loci), p53, exon 5, p53, exon 7, and p53, exon 8) in duplicate (for a total of 14 amplifications for each locus). Seven separate PCRs (33 cycles each) are run in duplicate using primers directed to detect fragments in the sample having 200 base pairs or more. Amplified DNA is placed on a 4% Nusieve (FMC Biochemical) gel (3% Nusieve, 1% agarose), and stained with ethidium bromide (0.5 μg/ml). The resulting amplified DNA is graded based upon the relative intensity of the stained gels. Samples from a patient with cancer or adenoma are detected as a band having significantly greater intensity than the bands associated with samples from patients who do not have cancer or precancer. Patients are identified as having cancer or adenoma by determining the amount of amplifiable DNA 200 base pairs or greater in length. The results are the same regardless of which locus is amplified.

### EXAMPLE 3

A patient, aged 50 years, presenting no symptoms of colorectal cancer and being at average risk for colorectal cancer, undergoes colonoscopy screening. The colonoscopy screening reveals no colorectal legions. The patient thereafter undergoes yearly colorectal screening by DNA analysis of stool samples from the patient, as described above in Example 1 or 2. In year 8, a genetic mutation is detected in patient's stool sample. Patient then undergoes a colonoscopy to detect the presence of colorectal lesions. During the procedure, polyps are detected and removed from the colon of the patient. The patient thereafter is monitored by undergoing colorectal screening every six months by DNA analysis of stool samples from the patient.

### EXAMPLE 4

A patient, aged 40 years, with symptoms or history indicating that a colonoscopy should be performed, undergoes colonoscopy screening. The colonoscopy screening reveals colorectal lesions. Based upon the colonoscopy results, the colorectal lesions are surgically removed from the colon of the patient. The patient thereafter is monitored by undergoing colorectal screening every six months by DNA analysis of stool samples from the patient, as described above in Example 1 or 2.

The invention has been described in terms of its preferred embodiments. Alternative embodiments are apparent to the skilled artisan upon examination of the specification and claims.

What is claimed is:

1. A method of screening for disease in a patient, comprising the steps of:
(a) performing a diagnostic procedure on a patient to detect characteristics indicative of the presence of a disease; and
(b) performing, at a predetermined time following said step (a), a non-invasive or minimally invasive genetic assay on a sample from the patient to detect a characteristic indicative of the presence of said disease in said sample.

2. The method of claim 1, wherein said disease is cancer or pre-cancer.

3. The method of claim 2, wherein said cancer or pre-cancer is colorectal cancer.

4. The method of claim 2, wherein said cancer or pre-cancer is selected from the group consisting of lung cancer, esophageal cancer, prostrate cancer, stomach cancer, pancreatic cancer, liver cancer, kidney cancer, bladder cancer, gallbladder cancer, uterine cancer, ovarian cancer, testicular cancer and lymphoma.

5. The method of claim 2, wherein said pre-cancer is adenoma.

6. The method of claim 1, wherein said diagnostic procedure is selected from the group consisting of sigmoidoscopy, mammography, prostate-specific antigen, carcinoembryonic antigen 1-18, digital rectal examination, breast self-examination, CT scan, virtual colonoscopy, MRI, PET scan, and colonoscopy.

7. The method of claim 1, wherein said diagnostic procedure is an invasive or substantially invasive diagnostic procedure.

8. The method of claim 1, wherein said diagnostic procedure is a non-genetic diagnostic procedure.

9. The method of claim 1, wherein said assay is selected from the group consisting of enumerated LOH, DNA integrity assay, mutation detection, expression assays, and FISH.

10. The method of claim 1, wherein said assay detects mutations at a genetic locus selected from the group consisting of p53, ras, APC, DCC, and BAT-26.

11. The method of claim 1, wherein said sample is a biological sample.

12. The method of claim 11, wherein said biological sample is selected from the group consisting of stool, urine, saliva, seminal fluid, blood, sputum, cerebrospinal fluid, pancreatic fluid, bile, lymph, pus, and aspirate.

13. The method of claim 11, wherein said biological sample substantially comprises cells and cellular debris.

14. The method of claim 1, wherein said step (b) is repeated at least once at a predetermined time interval.

15. The method of claim 1, wherein said step (b) is repeated at predetermined, substantially equal time intervals.

16. The method of claim 1, wherein said step (b) is repeated at substantially annual time intervals.

17. A method of monitoring cancer in a patient comprising the steps of:
(a) detecting characteristics indicative of the presence of cancer by performing a diagnostic procedure on a patient; and
(b) performing, at a predetermined time following said step (a), a non-invasive or minimally invasive genetic assay on a sample from the patient to detect characteristics indicative of the presence of cancer.

18. A method of monitoring cancer in a patient currently undergoing treatment or having undergone treatment for cancer, comprising the steps of:
(a) performing a diagnostic procedure on a patient to detect characteristics indicative of the presence of cancerous or precancerous cells; and
(b) performing, at a predetermined time following said step (a), a non-invasive or minimally invasive genetic assay on a sample from the patient to detect characteristics indicative of the presence of cancerous or precancerous cells.

19. A method for screening for indicia of cancer in a stool sample, comprising the steps of:
(a) performing a diagnostic procedure on a patient to detect characteristics indicative of the presence of a cancerous or precancerous lesion; and
(b) performing a non-invasive or minimally invasive genetic assay to detect the presence of cells or cellular debris from a cancerous or precancerous lesion into a voided stool sample.
